(19) United States
(12) Patent Application Publication
(10) Pub. No.: US 2006/0167005 A1
(43) Pub. Date: Jul. 27, 2006

(54) New therapeutic uses of (4-(2-fluorophenyl)-6-methyl-2-(1-piperazinyl) thieno [2,3-d] pyrimidine

(76) Inventors: David Cavalla, Cambridge (GB); Robert William Gristwood, Cambridge (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/525,532

(22) PCT Filed: Aug. 28, 2003
(86) PCT No.: PCT/GB03/03720

§ 371 Date: Jul. 25, 2005

(30) Foreign Application Priority Data
Aug. 29, 2002 (GB) 0220064.0
Jul. 9, 2003 (GB) 0316115.5

(51) Int. Cl. A61K 31/519

(52) U.S. Cl. 514/252.16

(57) ABSTRACT

(4-(2-Fluorophenyl)-6-Methyl-2-(1-Piperazinyl)Thieno[2,3-D]Pyrimidine or a salt thereof has value in the treatment of fibromyalgia, obesity, weight gain and other conditions.

### FIELD OF THE INVENTION

This invention relates to new uses for a known compound.

### BACKGROUND OF THE INVENTION

A number of non-tricyclic antidepressants have recently been developed that diminish the cardiovascular and anticholinergic liability characteristic of tricyclic antidepressants. These agents include those which inhibit uptake of serotonin and or noradrenaline. A number of uses has been proposed for these agents including the treatment of obesity and weight gain, Parkinson's disease, epilepsy, schizophrenia, obsessive compulsive disorder, substance abuse and drug addiction, pre-menstrual syndrome, eating disorders and migraines and for the encouragement of smoking cessation. Not all non-tricyclic antidepressants work in all disease/conditions and the relative merits of noradrenaline uptake inhibition to serotonin uptake inhibition for each disease/condition is not clear.

(4-(2-Fluorophenyl)-6-methyl-2-(1-piperazinyl)thieno[2,3-D]pyrimidine monohydrate hydrochloride is known (see U.S. Pat. No. 4,695,568). It has both serotonin and noradrenergic reuptake blocking properties, but also has important 5HT-3 receptor blocking activity, which would be expected to modify the pharmacological actions of the compound in vivo in a non-predictable manner. The utility of this compound in the treatment of pain, of urinary disorders, and of functional bowel disorders has recently been described in WO 02/094249, WO 03/063873 and PCT/GB03/02974, respectively (none published before the first priority date claimed in this case).

### SUMMARY OF THE INVENTION

Surprisingly, it has been found that the known compound identified above (referred to herein as MCI-225) can have valuable activity in the treatment of obesity and weight gain, Parkinson's disease, epilepsy, schizophrenia, obsessive-compulsive disorder, substance abuse, tobacco smoking (encouraging cessation), pre-menstrual syndrome, eating disorders, migraines, recovery from stroke, fibromyalgia, fatigue, nausea, vomiting and emesis including that produced by cancer chemotherapy and radiation therapies. Its combination of serotonin and noradrenergic reuptake blockade and 5HT-3 receptor blockade has not previously been clearly identified as being responsible for these activities. It will be appreciated that any suitable form of the active principle may be used, e.g. another salt form, or a prodrug or active metabolite.

### DESCRIPTION OF PREFERRED EMBODIMENTS

By means of this invention, the diseases/conditions outlined above can be treated, e.g. controlled or prevented. A particular embodiment of the invention is in the treatment of fibromyalgia, a chronic condition characterised by fatigue and widespread pain in muscles, ligaments and tendons. This condition was previously known by other names such as fibrositis, chronic muscle pain syndrome, psychogenic rheumatism and tension myalgia.

Another embodiment of the invention lies in a method for treating obesity or weight gain. This means reduction of weight, relief from being overweight, relief from gaining weight, or relief from obesity; all of which are usually due to extensive consumption of food.

Yet another embodiment of the invention lies in a method of treating Parkinson's disease. This means relief from the symptoms of Parkinson's disease which include, but are not limited to, slowly increasing disability in purposeful movement, tremors, bradykinesia, rigidity, and a disturbance of posture in humans.

Yet a further embodiment of the invention lies in a method treating fatigue, including that associated with cancer patients resulting from the disease and/or its treatment, in patients with chronic liver disease including chronic hepatitis C and in patients with chronic fatigue syndrome.

Further embodiments lie in the treatment of obsessive-compulsive disorder, substance abuse, pre-menstrual syndrome, eating disorders and migraine. These terms are used herein in a manner consistent with their accepted meanings in the art. See, e.g. Diagnostic and Statistical Manual of Mental Disorders 4^{th} Ed, American Psychiatric Association (1997).

The terms “method of treating or preventing,” “method of treating” and “method of preventing” may be used herein in connection with the disorders to which the invention relates. These terms mean the amelioration, prevention or relief from the symptoms and/or effects associated with these disorders, and are included within the scope of this invention.

For the purposes of this invention, the active compound can be formulated in any suitable manner together with a conventional diluent or carrier. The active compound is preferably administered by the oral route; other suitable routes of administration include sublingual/buccal, transdermal, intramuscular, intranasal, rectal, parenteral, subcutaneous, pulmonary and topical. An effective dose of the active agent will depend on the nature and degree of the complaint, the age and condition of the patient and other factors known to those skilled in the art. A typical daily dosage may be 0.1 mg to 5 g.

A pharmaceutical composition containing the active ingredient may be in the form of a sublingual tablet or patch. Suitable compositions for oral use include tablets, troches, lozenges, aqueous or oily suspensions, dispersible powders or granules, emulsions, hard or soft capsules, syrups and elixirs. Suitable additives include sweetening agents, flavouring agents, colouring agents and preserving agents. Tablets contain the active ingredient in admixture with non-toxic pharmaceutically acceptable excipients, e.g. inert diluents such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated, to form osmotic therapeutic tablets for controlled release. Hard gelatin capsules may include an inert solid diluent, for example calcium carbonate, calcium phosphate or kaolin; soft gelatin capsules may include water or an oil medium, for example peanut oil, liquid paraffin or olive oil.

The following Methods are given as examples to illustrate how the beneficial actions of MCI-225 may be demonstrated. Evidence provided in the three recent PCT publications/applications, to which reference is made above, may also be relevant.

Treatment of Obesity and Weight Gain

MCI-225 is evaluated in adult female obese Zucker rats over a period of 32 days. A control group of 6 animals is dosed daily with vehicle alone whilst a second group of 6 weight-matched animals receives MCI-225 at 30 mg/kg given orally once daily. Food is available ad libitum, except on days 0, 7, 14, 21, 28 and 32 when food was removed from the animals at 7.30 am and animals weighed within 2 hours following removal of food. Food is supplied after weights of animals are measured. A beneficial effect is demonstrated by the lower body weights of the MCI-225-treated animals.

Treatment of Substance Abuse/Drug Addiction

The effects of MCI-225 are determined in alcohol-preferring rats. Because of their pattern of drinking, these animals seem to represent a valid model of the human condition of alcoholism (McBride et al, 1990, Alcohol 7:199-205, Lankford et al, 1991, Pharmacol. Biochem. Behav., 8:293-299). After maximally preferred alcohol concentrations had stabilised for 4 days, MCI-225 at 30 mg/kg/day orally or vehicle was administered over 4 consecutive days. A beneficial effect of MCI-25 treatment is demonstrated by the reduction in intake of alcohol in terms of absolute g/kg and/or proportion of alcohol to total fluid intake.

Cessation of Smoking

The effects of MCI-225 are investigated in a model of nicotine withdrawal using the acoustic startle reflex in rats (see e.g. Helton et al, 1997, Neuropharmacology 36 (11-12):1511-1516). Nicotine (6 mg/kg/day) is administered for 12 days subcutaneously by osmotic minipumps. After 12 days, the pumps are removed and the animals allowed to go through spontaneous withdrawal. Cessation of chronic nicotine exposure leads to increased startle responses (sensorimotor reactivity) for 4 days following withdrawal. A beneficial of MCI-225 treatment, for example at 30 mg/kg/day following nicotine withdrawal, is demonstrated by the attenuation of the enhanced auditory startle response following withdrawal of nicotine.

Treatment of Stroke

The effects of MCI-225 are studied in a transient middle cerebral artery occlusion model in rats (see Chen et al, 1999, J. Neurol. Sci. 171(1):24-30). In particular, effects on an array of functional measures are studied, including rotarod, adhesive-backed somatosensory and neurological scores. A beneficial effect of treatment with MCI-225, at 30 mg/kg administered for example 2 hours after onset of occlusion, is demonstrated by improvement in one or more of the functional scores measured following ischaemia compared with vehicle-treated animals.

Treatment of Nausea/Emesis

The effects of MCI-225 are studied against cisplatin-induced emesis in the ferret (see Florczyk et al, 1982, Cancer Treat. Rep. 66(1):187-189). A beneficial effect of treatment with MCI-225, at 30 mg/kg orally given 1 hour prior to cisplatin administration, is demonstrated by a reduction in the emetic response compared with control animals. Efficacy against cisplatin predicts efficacy against radiation-induced nausea/vomiting. A wider spectrum of anti-emetic activity of MCI-225 may be demonstrated through the use of other emetogens including apomorphine in the ferret model.

1-15. (canceled)

16. A method for the treatment of a condition selected from the group consisting of obesity and weight gain; substance abuse and drug addition; smoking cessation; eating disorders, and obsessive-compulsive disorder; wherein the treatment comprises administering, to an individual in need of such treatment, (4-(2-fluorophenyl)-6-methyl-2-(1-piperazinyl)thieno[2,3-D]pyrimidine or a salt thereof.

17. The method, according to claim 16, wherein said condition is obesity and weight gain.

18. The method, according to claim 16, wherein said condition is substance abuse and drug addiction.

19. The method, according to claim 16, wherein smoking cessation is desired.

20. The method, according to claim 16, wherein said condition is an eating disorder.

21. The method, according to claim 16, wherein said condition is obsessive-compulsive disorder.

22. The method, according to claim 16, wherein the salt is the hydrochloride monohydrate.

23. A method for the treatment of a condition selected from the group consisting of pre-menstrual syndrome; migraine; nausea and vomiting; chemotherapy or radioactivity-induced emesis; and fatigue; wherein the treatment comprises, administering to an individual in need of such treatment, (4-(2-fluorophenyl)-6-methyl-2-(1-piperazinyl)thieno[2,3-D]pyrimidine or a salt thereof.

24. The method, according to claim 23, wherein said condition is pre-menstrual syndrome.

25. The method, according to claim 23, wherein said condition is migraine.

26. The method, according to claim 23, wherein said condition is nausea and vomiting.

27. The method, according to claim 23, wherein said condition is chemotherapy or radioactivity-induced emesis.

28. The method, according to claim 23, wherein said condition is fatigue.

29. The method, according to claim 23, wherein the salt is the hydrochloride monohydrate.

30. A method for the treatment of a condition selected from the group consisting of fibromyalgia; Parkinson's disease; stroke; and schizophrenia; wherein the treatment comprises administering, to an individual in need of such treatment, (4-(2-fluorophenyl)-6-methyl-2-(1-piperazinyl)thieno[2,3-D]pyrimidine or a salt thereof.

31. The method, according to claim 30, wherein said condition is fibromyalgia.

32. The method, according to claim 30, wherein said condition is Parkinson's disease.

33. The method, according to claim 30, wherein said condition is a stroke.

34. The method, according to claim 30, wherein said condition is schizophrenia.

35. The method, according to claim 30, wherein the salt is the hydrochloride monohydrate.
